SEQUENCE LISTING (2 sequences)

SEQ ID NO: 1 - protein, 116 aa, Artificial Sequence
  Synthetic construct.  “Variable region of the PDL humanizedanti-Tac antibody heavy chain.”
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 2 - protein, 106 aa, Artificial Sequence
  Synthetic construct.  “Variable region of the PDL humanizedanti-Tac antibody light chain.”
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

(19) United States
(12) Patent Application Publication
(10) Pub. No.: US 2010/0055098 A1
(43) Pub. Date: Mar. 4, 2010

(54) METHOD FOR TREATING MULTIPLE SCLEROSIS PATIENTS WITH ANTI-IL2R ANTIBODIES

(75) Inventor: Randy R. Robinson, Mountain View, CA (US)

Correspondence Address: Facet Biotech Corporation, ATTN; Legal Department, 1400 Seaport Blvd., Redwood City, CA, 94063, US

(73) Assignee: FACET BIOTECH CORPORATION, Redwood City, CA (US)

(21) Appl. No.: 12/549,501

(22) Filed: Aug. 28, 2009

Related U.S. Application Data
(60) Provisional application No. 61/190,362, filed on Aug. 28, 2008.

(51) Int. Cl. A61K 39/395

(52) U.S. Cl. 424/133.1; 424/158.1

(57) ABSTRACT

Methods are disclosed for using anti-IL-2R antibodies for the treatment of MS patients. In certain embodiments, the patients have neutralizing antibodies to IFN-beta.

### 1. CROSS REFERENCE TO RELATED APPLICATIONS

This application claims benefit under 35 U.S.C. §119(e) to application Ser. No. 61/190,362, filed on Aug. 28, 2008, the contents of which are incorporated herein in their entirety by reference.

### 2. STATEMENT REGARDING FEDERALLY SPONSORED RESEARCH

Not applicable.

### 3. BACKGROUND

Multiple sclerosis (MS) is a chronic inflammatory demyelinating disease of the central nervous system proposed to be mediated by autoreactive T cells (Compston and Coles, 2002, Lancet, 359 (9313):1221-1231). Activation, proliferation, and migration of these autoreactive T cells results in acute inflammatory attacks on oligodendrocytes. These acute inflammatory attacks and the resulting demyelination process present as sporadic lesions in the central nervous system, which can be detected subclinically as lesions by magnetic resonance imaging (MRI) and as clinically symptomatic, transient, neurological disabilities. Cumulative damage from these attacks results in chronic structural changes in axons, which manifest as permanent disabilities that accumulate with progressive disease (Compston and Coles, 2002, Lancet, 359 (9313):1221-1231).

Goals of MS treatment include preventing permanent disabilities and delaying disease progression. Short term therapeutic goals include reducing the relapse rate. IFN-beta is the most commonly used chronic maintenance agent for treating MS. Other agents used to treat MS, including corticosteroids, glatiramer acetate, mitoxantrone, and natalizumab, are only partially effective in managing clinical relapses, and some carry significant safety risks. Accordingly, it is important to identify additional agents that can be used to treat MS, alone or in combination with existing treatment modalities.

### 4. SUMMARY

IFN-beta is the most common first-line treatment for relapsing-remitting multiple sclerosis. Treatment with IFN-beta can result in the formation of neutralizing and binding antibodies against itself (see, e.g., Sorensen, 2003, Lancet, 362:1184-91). Several studies suggest that the presence of neutralizing antibodies reduces the efficacy of IFN-beta during treatment of multiple sclerosis (see, e.g., Sorensen, 2003, Lancet, 362:1184-91; Malucchi et al., 2004, Neurology, 62:2031-2037; Kappos et al., 2005, Neurology, 65:40-47). It is therefore desirable to develop methods for treating individuals having IFN-beta neutralizing antibodies.

Daclizumab has shown promise in clinical trials when administered concurrently with IFN-beta. Results from two open-label studies of daclizumab given to patients with relapsing forms of MS indicate that daclizumab is well tolerated and reduces the number of new MRI lesions in patients that do not respond, or respond poorly to IFN-beta therapies (Bielekova, et al., 2004, Proc Natl Acad Sci USA, 101 (23):8705-8708; Rose, 2003, Proceedings of the 55^{th} Annual Meeting of the American Academy of Neurology, 60 (suppl.1):A478-A479 (abstract); and Rose et al., 2004, Annals of Neurology, 56 (6):864-7). Neither of these trials determined the percentage of poorly responding or non-responding patients having neutralizing antibodies to IFN-beta and whether daclizumab would be an effective treatment option for patients having neutralizing antibodies to IFN-beta.

The methods described herein relate to the findings of a randomized, double blind, placebo controlled, trial designed to evaluate the efficacy of daclizumab. Patient serum samples were collected over the course of the trial and used to determine the percentage of subjects that tested positive for neutralizing antibodies to IFN-beta. The incidence of subjects that were positive for neutralizing antibodies to IFN-beta was 9/68 (13%) in the placebo group, 7/63 (11%) for the 1 mg/kg daclizumab group, and 7/65 (11%) for the 2 mg/kg daclizumab group. In subjects that tested positive for neutralizing antibodies to IFN-beta, both the 1 mg/kg and 2 mg/kg daclizumab groups had significantly fewer new or enlarged gadolinium contrast-enhanced MRI lesions (Gd-CEL) compared to the placebo group (see Example 1). In both daclizumab treatment groups, the mean number of new or enlarged Gd-CEL lesions was lower in subjects with neutralizing antibodies to IFN-beta compared to those who were negative for neutralizing antibodies to IFN-beta (see Example 1).

These results indicate that daclizumab is an effective drug for treating MS patients with neutralizing antibodies to IFN-beta. Accordingly, the methods described herein disclose the use of daclizumab for treating MS patients having neutralizing antibodies to interferon beta. In some embodiments, the method includes administering a therapeutically effective amount of daclizumab to a subject, thereby reducing or stabilizing disease progression and treating the subject. Symptoms of MS that can be stabilized or improved using the methods described herein include, but are not limited to, reducing the relapse rate, stabilizing or reducing the rate of disability progression as measured by standard scores such as the Expanded Disability Status Scale (EDSS) score, decreasing the number of new or enlarged T1 gadolinium contrast-enhanced MRI lesions (Gd-CEL), and/or decreasing the number of new or enlarged T2 lesions. The subject being treated can have relapsing/remitting MS, secondary progressive MS, progressive relapsing MS, or primary progressive MS. In addition to daclizumab, other IL-2R antibodies, such as monoclonal antibodies, chimeric antibodies, humanized antibodies, or fully human antibodies that specifically bind to the alpha or p55 (Tac) chain of the IL-2 receptor can be used in the methods described herein.

It is to be understood that both the foregoing general description and the following detailed description are exemplary and explanatory only and are not restrictive of the compositions and methods described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting.

### 5. BRIEF DESCRIPTION OF THE FIGURES

FIG. 1 depicts the study design for the randomized, double-blind placebo-controlled study of daclizumab in patients with MS; and,

FIG. 2 depicts the total number or new or enlarged lesions sub-grouped by treatment and IFN-beta neutralizing antibody status.

### 6. DETAILED DESCRIPTION

Described below are methods for treating multiple sclerosis. The methods of the invention involve the administration of an anti-IL-2R antibody, preferably an antibody that inhibits the interaction between IL-2 and its high affinity receptor CD25.

The methods described herein are based upon the discovery that anti-IL-2R antibodies are useful for treating MS patients having neutralizing antibodies to IFN-beta. In particular, the methods can be used to ameliorate one or more symptoms associated with disease progression in various forms of MS.

### 6.1 Definitions

Relapsing-remitting multiple sclerosis: By “relapsing-remitting multiple sclerosis” (or “RRMS”) herein is meant a clinical course of MS that is characterized by clearly defined, acute attacks with full or partial recovery and no disease progression between attacks.

Secondary-progressing multiple sclerosis: By “secondary-progressive multiple sclerosis” (“SPMS”) herein is meant a clinical course of MS that initially is relapsing-remitting, and then becomes progressive at a variable rate, possibly with an occasional relapse and minor remission.

Progressive relapsing multiple sclerosis: By “progressive relapsing multiple sclerosis” (“PRMS”) herein is meant a clinical course of MS that is progressive from the onset, punctuated by relapses. There is significant recovery immediately following a relapse, but between relapses there is a gradual worsening of disease progression.

Primary progressive multiple sclerosis: By “primary progressive multiple sclerosis” (“PPMS”) herein is meant a clinical course of MS that presents initially in the progressive form with no remissions.

Antibody: As used herein, “antibody” refers to an immunoglobulin molecule immunologically reactive with a particular antigen, and includes both polyclonal and monoclonal antibodies. The term includes genetically engineered forms, such as chimeric antibodies and heteroconjugate antibodies, antigen binding forms of antibodies (e.g., Fab′, F(ab′)₂, Fab, Fv and rIgG), recombinant single chain Fv fragments (scFv), bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301. An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen. Methods for producing and screening for specific antibodies using hybridoma technology are routine and well known in the art. In a non-limiting example, mice can be immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well-known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Antibody Structure: Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

“V_{H}” and “V_{L}”: References to “V_{H}” refer to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab.

“Single chain Fv” or “scFv”: The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

“Epitope” or “antigenic determinant”: “Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The determination of whether two antibodies bind substantially to the same epitope is accomplished by the methods known in the art, such as a competition assay. In conducting an antibody competition study between a control antibody (for example, daclizumab) and any test antibody, one can first label the control antibody with a detectable label, such as, biotin, enzymatic, radioactive label, or fluorescent label to enable the subsequent identification. The intensity of the bound label is measured. If the labeled antibody competes with the unlabeled antibody by binding to an overlapping epitope, the intensity will be decreased relative to the binding by negative control unlabeled antibody.

“Monoclonal Antibody”: The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563 681 (both of which are incorporated herein by reference in their entireties).

“Chimeric Antibody”: A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Any of the anti-IL-2R antibodies described herein can be chimeric.

“Humanized antibody” or “humanized immunoglobulin”: The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least 90%, and at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. 91:969 973 (1994), and chain shuffling (U.S. Pat. No. 5,565,332), all of which are hereby incorporated by reference in their entireties. The anti-IL-2R antibodies described herein include humanized antibodies, such as mouse humanized antibodies, fully human antibodies, and mouse antibodies.

### 6.2 Patient Population

6.2.1 Multiple Sclerosis Disease State

Techniques such as magnetic resonance imaging, spectroscopy and electrophysiological techniques can be used to stage the disease in a patient. Such techniques may be employed to assess whether a therapeutic regimen of the invention (entailing the administration of an anti-IL-2R antibody alone or in combination therapy) should be initiated. The earliest detectable event in the development of a new lesion is an increase in permeability of the blood-brain barrier associated with inflammation (McDonald, 1994, J. Neuropathol. Exp. Neurol. 53 (4):338-43). Generally, once such a lesion is detected, a patient can undergo treatment with an anti-IL-2R antibody.

A patient suitable for the present methods can have any of the four states of MS identified in Section 6.1 above (i.e., RRMS, SPMS, PRMS, or PPMS). In certain aspects, the patient has RRMS or PRMS and is in a state of remission at the time anti-IL-2R therapy is initiated. In other aspects, the patient has RRMS or PRMS and is in a state of relapse at the time anti-IL-2R therapy is initiated.

6.2.2 Interferon-Beta Neutralizing Antibody Status

In some embodiments, a therapeutically effective amount of an anti-IL-2R antibody is administered to an IFN-beta NAb positive patient in the absence of treatment with an IFN-beta product.

The anti-IL-2R antibodies described herein find use in treating MS patients having neutralizing antibodies to INF-beta. Anti-IFN-beta antibodies typically are identified by specific binding antibody assays, and if detected, are called binding antibodies (BAbs). BAbs are present in a high percentage of MS patients and can occur at low levels without apparent clinical problems. Because BAb assays do not measure the ability of these antibodies to interfere with the biological function of IFN-beta, assays that measure the ability of BAbs to neutralize IFN-beta's effect in vitro can be used. Neutralizing antibodies (NAbs) to IFN-beta are clinically relevant because they can reduce the therapeutic benefits associated with IFN-beta treatment as measured by the frequency and rate of relapse, MRI activity and changes in the Expanded Disability Status Scale score. For example, a comparison between NAb negative and persistent IFN-beta NAb positive MS patients demonstrated that IFN-beta NAb positive patients had a higher mean relapse rate, a higher risk of sustained progression, and a lower probability of being relapse free (see, e,g, Malucchi, et al., 2004, Neurology, 62: 2031-2037).

As used herein antibodies that bind to IFN-beta but do not neutralize its activity are referred to as “binding antibodies (i.e. BAbs). Serum samples from MS patients can be screened for the existence of BAbs to IFN-beta by ELISA as described in Kappos et al. (Kappos et al., 2005, Neurology, 65: 40-47). Serum samples with a positive result on ELISA can be screened for neutralizing antibodies to IFN-beta. As used herein, “neutralizing antibodies to IFN-beta” are defined as antibodies that interfere with the biological activity of IFN-beta as measured, for example, using an assay that can detect one or more IFN-beta inducible genes and/or their products. Examples of assays suitable for detecting IFN-beta NAbs include assays that detect oligo-A-synthetases, neopterin, beta-2 microglobulin, interleukin-10, soluble vascular cell adhesion molecule and myxovirus A (MxA) (Pachner et al., 2003, Neurology, 61 (Suppl 5): S24-S26).

In some embodiments, IFN-beta neutralizing activity is detected using an antiviral cytopathic effect (CPE) assay (Kappos et al., 2005, Neurology, 65: 40-47). This assay is recommended by the World Health Organization (WHO) (Malucchi et al., 2004, Neurology, 62: 2031-2037). Based on WHO recommendations, data from the CPE neutralization assay are reported as the reciprocal of the highest dilution of serum inducing 50% neutralization (i.e., neutralizing 10 U/mL of IFN-beta activity to an apparent 1 U/mL of activity) (Malucchi et al., 2004, Neurology, 62: 2031-2037). The neutralization titer of a serum sample is calculated according to Kawade's formula and is expressed in Laboratory Units (LU). Typically, a level of ≧20 LU/mL is considered the threshold for positive (Malucchi et al., 2004, Neurology, 62: 2031-2037).

The neutralization titer of a serum sample can be used to categorize MS patients according to IFN-beta NAb status (see, e.g., Malucchi et al., 2004, Neurology, 62: 2031-2037; Kappos et al., 2005, Neurology, 65:40-47; Farrell, et al., 2008, Multiple Sclerosis, 14:212-218). At least two categories MS patients can be distinguished: IFN-beta NAb negative and IFN-beta NAb positive. Depending on the assay used to measure IFN-beta NAb, the titer at which a patient is considered NAb positive can vary. Typically, IFN-beta NAb negative patients have an IFN-beta NAb titer between 0 to <20 and IFN-beta NAb positive patients have an IFN-beta NAb titer of ≧20 in one or more samples collected at selected time intervals.

In some embodiments, a standard cell-based viral inhibition assay performed by Athena Diagnostics, Worcester, Mass., following the guidelines of the NIH Committee on Human Antibodies to Interferon and the World Health Organization for the standardization of interferon neutralization bioassays is used. Titer is defined in the assays run by Athena Diagnostics as “the reciprocal of the dilution of patient serum which reduce interferon activity by a standard amount” (Grossberg et al., 1988, J Interferon Research, 8: 5-7).

In some embodiments, IFN-beta NAb positive patients have a titer ≧25 and IFN-beta NAb negative patients have a titer <25.

In some embodiments, an additional category of IFN-beta NAb MS patients can be identified, indeterminate, which is characterized by ≧1 NAb titers of 5 to 19 or one, but not two consecutive of ≧20 (Kappos et al., 2005, Neurology, 65:40-47).

The determination of NAb status generally requires that more than one sample be taken from a patient at selected times. The determination of sampling time is not critical to the methods described herein. For example, NAb status can be monitored before, during and after initiation of treatment with IFN-beta. In this example, sampling times can be selected by a medical practitioner based in part on the length of time a patient has received IFN-beta treatment, which IFN-beta product the patient has been treated with, the approximate time frame in which BAbs and NAbs are predicted to appear in treated individuals, and whether the patient is showing one or more of the following symptoms: increased relapse rate, an increase in the Expanded Disability Status Scale score, an increased number of T1 Gd-CEL, and increase in new or enlarged T2 MRI lesions. See, e.g., Perini et al., 2004, J Neurology, 251:305-309; Sorensen, et al., 2003, Lancet, 362:184-191; Pachner, 2003, Neurology, 61 (Suppl 5):S2-S5; Perini et al., 2004, J Neurology, 251:305-309; and Farrell, et al., 2008, Multiple Sclerosis, 14:212-218.

By way of another example, IFN-beta NAb status can be monitored prior to the initiation of treatment with an anti-IL-2R antibody and at selected intervals (e.g., monthly, once every two months, once every three months, once every six months) during treatment with an anti-IL-2R antibody.

By way of another example, IFN-beta NAb status can be monitored prior to the initiation of treatment with an IFN-beta product and an anti-IL-2R antibody and at selected intervals (e.g., monthly, once every two months, once every three months, once every six months) during concomitant treatment with an IFN-beta product and an anti-IL-2R antibody.

MS patients that are not doing well on or failing to respond to INF-beta, and are IFN-beta NAb positive can be treated with a therapeutically effective amount of an anti-IL-2R antibody. MS patients that are responding poorly to IFN-beta treatment and are IFN-beta NAb positive generally have a higher mean relapse rate, a higher risk of experiencing a second relapse, a higher risk of having a sustained progression of ≧1 on EDSS, and a lower probability of being relapse free (Malucchi et al., 2004, Neurology, 62: 2031-2037). Accordingly, a number of clinical endpoints can be used to determine whether a patient is responding to IFN-beta including the frequency and rate of relapse, a 1 point or greater increase in the Expanded Disability Status Scale (EDSS) score, an increase in the number of T1 gadolinium contrast-enhanced lesions (Gd-CEL), and/or an increase in the number of new or enlarged T2 MRI lesions.

The data required to determine clinical endpoints can be collected at the start of IFN-beta treatment and/or during follow-up visits. For example, relapses are typically assessed by history and physical examination defined as the appearance of a new symptom or worsening of an old symptom attributable to multiples sclerosis, accompanied by an appropriate new neurological abnormality or focal nuerological dysfunction lasting at least 24 hours in the absence of fever, and preceded by stability or improvement for at least 30 days (see, e.g., Sorensen et al., 2003, Lancet, 362: 1184-1191.

By way of another example, the failure of an IFN-beta NAb positive MS patient to respond as predicted to interferon-beta treatment can be measured using Magnetic Resonance Imaging (MRI). MRI is a noninvasive diagnostic technique that produces computerized images of internal body tissues and is based on nuclear magnetic resonance of atoms within the body induced by the application of radio waves. Brain MRI is an important tool for understanding the dynamic pathology of multiple sclerosis. T₂-weighted brain MRI defines lesions with high sensitivity in multiple sclerosis and is used as a measure of disease burden. However, such high sensitivity occurs at the expense of specificity, as T₂ signal changes can reflect areas of edema, demyelination, gliosis and axonal loss. Areas of gadolinium (Gd) enhancement demonstrated on T₁-weighted brain MRI are believed to reflect underlying blood-brain barrier disruption from active perivascular inflammation. Such areas of enhancement are transient, typically lasting <1 month. Gadolinium-enhanced T₁-weighted brain MRI are therefore used to assess disease activity. Most T₂-weighted (T2) lesions in the central white matter of subjects with multiple sclerosis begin with a variable period of T₁-weighted (T1) gadolinium (Gd) enhancement. T1 Gd-enhancing and T2 lesions represent stages of a single pathological process. Brain MRI is a standard technique for assessing T1 and T2 MRI lesions and is routinely used to assess disease progression in MS (e.g., see Lee et al., Brain 122 (Pt 7):1211-2, 1999).

By way of another example, functional system scores developed by Kurtzke and referred to herein as “Expanded Disability Status Scale (EDSS)” can be used to rate neurological impairment in MS patients (Kurtzke, 1983, Neurology, 33-1444-52). The EDSS comprises 20 grades from 0 (normal) to 10 (death due to MS), progressing in a single-point step from 0 to 1 and in 0.5 point steps upward. The scores are based on a combination of functional-system scores, the patient's degree of mobility, need for walking assistance, or help in the activities of daily living. The functional-system scores measure function within individual neurological systems including visual, pyramidal, cerebellar, brainstem, sensory, bowel and bladder, cerebral and other functions.

Typically, an MS patient is human, although non human subjects also can be treated with the methods described herein.

### 6.3 Anti-IL-2R Antibodies

As used herein an “anti-IL-2R antibody” is an antibody that specifically binds an IL-2 receptor. For example, in some embodiments, an anti-IL-2R antibody binds the high affinity IL-2 receptor (K_{d}≈10 pM). This receptor is a membrane receptor complex consisting of the two subunits: IL-2R-alpha (also known as T cell activation (TAC) antigen, CD25, or p55) and IL-2R-beta (also known as p75 or CD122). In other embodiments, an anti-IL-2R antibody binds the intermediate affinity IL-2 receptor (K_{d}=100 pM), which consists of the p75 subunit and a gamma chain. In other embodiments, an anti-IL-2R antibody binds the low affinity receptor (K_{d}=10 nM), which is formed by p55 alone.

Anti-IL-2R antibodies suitable for use in the methods described herein include monoclonal antibodies, chimeric antibodies, humanized antibodies, or fully human antibodies. Examples of anti-IL-2R antibodies capable of binding Tac (p55) include, but are not limited to, Zenapax®, the chimeric antibody basiliximab (Simulect®), BT563 (see Baan et al., Transplant. Proc. 33:224-2246, 2001), and 7G8, and HuMax-TAC being developed by Genmab. The mik-betal antibody specifically binds the beta chain of human IL-2R. Additional antibodies that specifically bind the IL-2 receptor are known in the art. For example, see U.S. Pat. No. 5,011,684; U.S. Pat. No. 5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559.

In some embodiments, the anti-IL-2 receptor antibody is Zenapax® (daclizumab). The recombinant genes encoding Zenapax® are a composite of human (about 90%) and murine (about 10%) antibody sequences. The donor murine anti-Tac antibody is an IgG2a monoclonal antibody that specifically binds the IL-2R Tac protein and inhibits IL-2-mediated biologic responses of lymphoid cells. The murine anti-Tac antibody was “humanized” by combining the complementarity-determining regions and other selected residues of the murine anti-TAC antibody with the framework and constant regions of the human IgG1 antibody. The humanized anti-Tac antibody daclizumab is described and its sequence is set forth in U.S. Pat. No. 5,530,101, see SEQ ID NO: 5 and SEQ ID NO: 7 for the heavy and light chain variable regions respectively. U.S. Pat. No. 5,530,101 and Queen et al., Proc. Natl. Acad. Sci. 86:1029-1033, 1989 are both incorporated by reference herein in their entirety.

Zenapax® has been approved by the U.S. Food and Drug Administration (FDA) for the prophylaxis of acute organ rejection in subjects receiving renal transplants, as part of an immunosuppressive regimen that includes cyclosporine and corticosteroids. Zenapax® has been shown to be active in the treatment of human T cell lymphotrophic virus type 1 associated myelopathy/topical spastic paraparesis (HAM/TSP, see Lehky et al., Ann. Neuro., 44:942-947, 1998). The use of Zenapax® to treat posterior uveitis has also been described (see Nussenblatt et al., Proc. Natl. Acad. Sci., 96:7462-7466, 1999).

In some embodiments, the antibody is basiliximab, marketed as Simulect® by Novartis Pharma AG. Simulect® is a chimeric (murine/human) antibody, produced by recombinant DNA technology, that functions as an immunosuppressive agent, specifically binding to and blocking the alpha chain of the IL-2R on the surface of activated T-lymphocytes.

Antibodies that bind the same (or overlapping) epitope as daclizumab or basiliximab can be used in the methods disclosed herein. As shown by Binder et al., 2007, Cancer Res. 67 (8):3518-23, the epitopes of daclizumab and basiliximab, are overlapping and map to a peptide string at positions 116 to 122 of CD25, the sequence of which is “ERIYHFV”. This epitope maps to the interaction site between IL-2 and CD25. In certain aspects, binding to the same or overlapping epitope as daclizumab or basiliximab can be identified in a competition assay. In specific embodiments, an anti-IL-2R antibody inhibits the binding daclizumab or basiliximab to CD25 or CD25-expressing cells by at least 50%, at least 60% or at least 75% in a competition assay, for a competition assay as described in “Epitope Mapping,” Chapter 11, in Using Antibodies by Ed Harlow and David Lane. Cold Spring Harbor Laboratory Press, Cold Spring Harbor, N.Y., USA, 1999.

An anti-IL-2R antibody suitable for the methods described herein can have at least 90%, at least 95%, at least 98%, or at least 99% sequence identity with daclizumab. An anti-IL-2R antibody suitable for the methods described herein can also have one, two, three, four, five or six CDRs with at least 80%, at least 85%, or at least 90% sequence identity with the corresponding CDRs of daclizumab.

An anti-IL-2R antibody suitable for the methods described herein can be of any isotype, including but not limited to, IgG1, IgG2, IgG3 and IgG4.

Preferably, an anti-IL-2R antibody is administered in the present methods in purified form. As, used herein, purified form means that the anti-IL-2R antibody is at least 30%, more preferably at least 40%, and yet more preferably at least 50% pure. In specific embodiments, the anti-IL-2R antibody is 60%, 70%, 80%, 90%, 95% or 98% pure.

### 6.4 Therapeutic Methods and Dosage Regimens

6.4.1 Clinical Benefits

The outcome of the therapeutic methods described herein is to produce in a patient at least one healthful benefit, which includes but is not limited to: prolonging the lifespan of a patient, prolonging the onset of symptoms of MS (for example by prolonging the onset of initial symptoms of MS and/or by prolonging the onset of relapses of MS), stabilizing or reducing the rate of disability progression, prolonging the onset of a more advanced stage of MS, and/or alleviating a symptom of the MS after onset of a symptom of MS. As used herein, “symptom” refers to any subjective or objective evidence of disease or of a subject's condition. Subjective evidence is typically evidence perceived by the subject, such as a noticeable change in a subject's condition indicative of some bodily or mental state. Objective evidence refers to any abnormality indicative of disease that is discoverable on examination or assessment of a subject, such as any parameter used to assess immunological status, the presence of lesions in a subject with multiple sclerosis.

As used herein, a “therapeutically effective dose” is a dose sufficient to prevent advancement, cause regression, or reduce one or more of the symptoms associated with disease progression in multiple sclerosis. For example, in some embodiments administration of a therapeutically effective dose of an anti-IL-2R antibody to an IFN-beta NAb positive MS patient decreases the number of relapses by at least one that occur in a given time period, such as 1 year, in the treated patient.

In other embodiments, administration of a therapeutically effective dose of an anti-IL-2R antibody to an IFN-beta NAb positive MS patient decreases the number and enlarged T1 gadolinium contrast-enhancing lesions (Gd-CEL) detected in the patient's brain. For example, as shown in Example 2, Table 6, treatment of IFN-beta positive MS patients with daclizumab reduced the mean number of new and enlarged T1 Gd-CEL lesions by 17 fold or greater.

In other embodiments, administration of a therapeutically effective dose of an anti-IL-2R antibody to an IFN-beta NAb positive MS patient decreases the number of new or enlarged T2 MRI lesions detected in the patient's brain.

In other embodiments, administration of a therapeutically effective dose of an anti-IL-2R antibody to an IFN-beta NAb positive MS patient stabilizes or slows the rate of a patient's disability progression as determined by EDSS.

In other embodiments, administration of a therapeutically effective dose of an anti-IL-2R antibody to an IFN-beta NAb positive MS patient reduces a patient's disability score by 10% to 75%. For example in some embodiments, a patient's disability score can be reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, or by at least 76%.

6.4.2 Treatment Period

Multiple sclerosis is a chronic inflammatory disease of the central nervous system and is associated with periods of disability (relapse) alternating with periods of recovery (remission), and often results in chronic progressive multiple sclerosis characterized by neurologic disability. According to a survey of physicians, there are four main categories of MS: Relapsing/Remitting (RRMS); Secondary Progressive (SPMS); Progressive Relapsing Multiple Sclerosis (PRMS); and Primary Progressive (PPMS) (see Lublin et al., 1996, Neurology 46:907-911). The therapeutic methods described herein can be practiced on any category of MS patient, and are preferably practiced on a patient with RRMS, SPMS or PRMS during peak periods of relapse. For example, in certain embodiments, an anti-IL-2R antibody is administered during a period of relapse in a patient with RRMS, SPMS or PRMS. In other embodiments, the anti-IL-2R antibody is administered during a period of remission in such a patient. In yet other embodiments, the anti-IL-2R antibody is administered during disease progression in a SPMS or PPMS patient.

6.4.3 Frequency of Administration

Single or multiple administrations of anti-IL-2R antibodies can be carried out with dosages and frequency of administration selected by the treating physician. For example, the treating physician can screen a patient diagnosed with MS for the presence of IFN-beta neutralizing antibodies for the development of a treatment plan. Generally, multiple doses are administered. For example, multiple administration of Zenapax® (daclizumab) or other anti-IL-2R antibodies can be utilized, such as administration monthly or every four weeks, bimonthly, every 8 weeks, every 7 weeks, every 6 weeks, every 5 weeks, every other week, weekly or twice per week.

The dosages can be adjusted upwards or downwards during the course of treatment, for example according to the patient's responsiveness, disease status and IFN-beta neutralizing antibody status.

An exemplary protocol for administration of Zenapax® (daclizumab), applicable to other anti-IL-2R antibodies, is described in the Examples section below. Treatment will typically continue for at least a month, more often for two or three months, sometimes for six months or a year, and indefinitely, i.e., chronically. Repeat courses of treatment are also possible.

In certain aspects, a patient is treated for a period of 56 weeks, a period of 44 weeks, a period of 36 weeks, a period of 30 weeks, a period of 24 weeks, a period of 20 weeks, or a period of 16 weeks.

Optionally, repeat treatment of a period of the same or a different duration can be administered, for example after a break from anti-IL-2R treatment for a period of up to 4 weeks, 8 weeks, 12 weeks, 16 weeks, 20 weeks, 30 weeks, 40 weeks, 1 year or longer.

6.4.4 Mode of Administration and Formulations

Anti-IL-2R antibodies can be administered parenterally, i.e., subcutaneously, intramuscularly or intravenously or by means of a needle-free injection device. The compositions for parenteral administration will commonly include a solution of an anti-IL-2R antibody in a pharmaceutically acceptable carrier. Pharmaceutically-acceptable, nontoxic carriers or diluents are defined as vehicles commonly used to formulate pharmaceutical compositions for animal or human administration. See, for example, Remington's Pharmaceutical Sciences, by E. W. Martin, Mack Publishing Co., Easton, Pa., 15th Edition (1975), for a description of compositions and formulations suitable for pharmaceutical delivery of the anti-IL-2R antibodies disclosed herein. See US Pat. Appl. Pub. Nos. 2003/0138417 and 2006/0029599 for a description of liquid and lyophilized formulations suitable for the pharmaceutical delivery of daclizumab.

Methods for preparing pharmaceutical compositions are known those skilled in the art (see Remington's Pharmaceutical Science, 15th ed., Mack Publishing Company, Easton, Pa., 1980). In addition, the pharmaceutical composition or formulation can include other carriers, adjuvants, or nontoxic, non-therapeutic, nonimmunogenic stabilizers and the like. Effective amounts of such diluent or carrier will be those amounts that are effective to obtain a pharmaceutically acceptable formulation in terms of solubility of components, or biological activity.

The concentration of antibody in the formulations can vary widely, i.e., from less than about 0.5%, usually at or at least about 1% to as much as 15 or 20% by weight or from 1 mg/mL to 100 mg/mL. The concentration is selected primarily based on fluid volumes, viscosities, etc., in accordance with the particular mode of administration selected.

6.4.5 Dosage

Generally a suitable dose of Zenapax® (daclizumab) is about 0.5 milligram per kilogram (mg/kg) to about 5 mg/kg, such as a dose of about 0.5 mg/kg, of about 1 mg/kg, about 1.5 mg/kg, about 2 mg/kg, about 2.5 mg/kg, about 3.0 mg/kg, about 3.5 mg/kg, about 4.0 mg/kg, about 4.5 mg/kg, or about 5.0 mg/kg administered intravenously or subcutaneously. Unit dosage forms are also possible, for example 50 mg, 100 mg, 150 mg, 200 mg, 300 mg, 400 mg, or up to 500 mg per dose.

Other dosages also can be used. It has been suggested that that serum levels of 2 to 5 μg/mL are necessary for saturation of the Tac subunit of the IL-2 receptor to block the responses of activated T lymphocytes, although higher levels, such as approximately 5 to 40 μg/mL, may be necessary for clinical efficacy. One of skill in the art will be able to construct an administration regimen to keep serum levels within the 2 to 40 μg/mL range.

Doses of basiliximab (Simulect®) are likely to be lower, for example 0.25 mg/kg to 1 mg/kg, e.g., 0.5 mg/kg, or unit doses of 10, 20, 40, 50 or 100 mg. The general principle of keeping the IL-2 receptor saturated can be used to guide the choice of dose levels of other IL-2R antibodies.

The dosages can be adjusted upwards or downwards during the course of treatment, for example according to the patient's responsiveness, disease status and IFN-beta neutralizing antibody status.

6.5 Combination Therapy

Described below are combinatorial methods and related compositions for treating multiple sclerosis. The combinatorial methods involve the administration of at least two agents to a patient, the first of which is an anti-IL-2R antibody, and the second of which is a second therapeutic agent.

The combinatorial therapy methods can result in a greater than additive effect, providing therapeutic benefits that are not observed when a single agent is used to treat MS.

The anti-IL-2R antibody and the second therapeutic agent can be administered concurrently or successively. As used herein, the anti-IL-2R antibody and the second therapeutic agent are said to be administered concurrently if they are administered to the patient on the same day, for example, simultaneously, or 1, 2, 3, 4, 5, 6, 7 or 8 hours apart. In contrast, the anti-IL-2R antibody and the second therapeutic agent are said to be administered successively if they are administered to the patient on the different days, for example, the anti-IL-2R antibody and the second therapeutic agent can be administered at a 1-day, 2-day or 3-day intervals. Administration of the anti-IL-2R antibody can precede or follow administration of the second therapeutic agent.

As a non-limiting example, the anti-IL-2R antibody and second therapeutic agent can be administered concurrently for a period of time, followed by a second period of time in which the administration of the anti-IL-2R antibody and the second therapeutic agent is alternated.

Because of the potentially synergistic effects of administering a anti-IL-2R antibody and a second therapeutic agent, such agents can be administered in amounts that, if one or both of the agents is administered alone, is/are not effective for treating MS.

Because MS is characterized by periods of disability (relapse) alternating with periods of recovery (remission), and eventually can result in chronic progressive multiple sclerosis, the combination therapy methods of the present invention can be administered during any of these periods, concurrently or in an alternating manner. A few non limiting embodiments of such modes of administration include by way of example, administration of the second therapeutic agent concurrently with the anti-IL-2R antibody. Such concurrent administration can take place during a period of relapse in multiple sclerosis, during a period of disease remission, or during chronic progressive phase of the disease. Alternatively, the second therapeutic agent and the anti-IL-2R antibody are administered successively. In such methods of successive administration, the second therapeutic agent can be administered prior to administration of the anti-IL-2R antibody or after administration of the anti-IL-2R antibody. The anti-IL-2R antibody and the second therapeutic agent can be administered successively during the same phase of the disease, for example during remission, relapse or chronic progressive phase of multiple sclerosis in a patient. Alternatively, the anti-IL-2R antibody and the second therapeutic agent can be administered successively at different phases of the disease. For example, the anti-IL-2R antibody can be administered during a period of relapse and the second therapeutic agent can administered during a period of remission in the same patient, or vice versa.

In certain embodiments, the second therapeutic agent is an immunosuppressive agent or a biological response modifier. Examples of immunosuppressive agents include, but are not limited to, cyclosporine, FK506, rapamycin, or prednisone.

Examples of biological response modifiers include, but are not limited to, interleukins (such as interleukin 4) or antibodies (e.g., an antibody to CCR1, RANTES, MCP-1, MIP-2, Interleukin-1α, Interleukin-1β, Interleukin-6, Interleukin-12, p35 or IFN-γ).

In a specific embodiment, the second therapeutic agent is IFN-beta. Examples of suitable INF-beta products include, but are not limited to, one of the three IFN-beta products that have been approved: IFN-beta-1b (Betaferon, Schering AG, Berlin, Germany), IFN-beta-1a (Avonex, Biogen, Cambridge Mass.), and IFN-beta-1a (Rebif, Ares-Serono, Geneva, Switzerland). In another embodiment, the second therapeutic agent is not IFN-beta.

In some embodiments, the second therapeutic agent is a second anti-IL-2R antibody. The antibodies can be administered concurrently, prior to or following administration of a first anti-IL-2R antibody.

### 7. EXAMPLES

### Example 1

### CHOICE Study

The CHOICE study was a Phase 2, randomized, double-blinded, placebo-controlled, multi-center study of subcutaneous (SC) daclizumab added to interferon (IFN)-beta in the treatment of active, relapsing forms of MS. A summary of the CHOICE study design is described below. Results from the CHOICE study confirmed that daclizumab at 2 mg/kg every two weeks, but not 1 mg/kg every four weeks, significantly decreased the number of new MRI lesions in patients who have active, relapsing forms of MS on concurrent IFN-beta therapy (Montalban, X. et al., Multiple Sclerosis, 13: S18-S18 Suppl. 2 OCTOBER 2007; and, Kaufman, M. D., et. a., Neurology, 70 (11): A220-A220 Suppl. 1 MAR. 11 2008).

The CHOICE study was a randomized, double-blinded, multi-center study comparing daclizumab and placebo as additional treatment for approximately 230 patients currently on IFN-beta therapy for active, relapsing forms of MS. Approximately 50 of these patients will undergo pharmacodynamic and pharmacokinetic testing. The dosing routes are subcutaneous (SC) for daclizumab and placebo, and SC or intramuscular (IM) for the concomitant IFN-beta regimen.

A patient is enrolled in the study once he or she has been randomized. Enrolled patients remained on their baseline IFN-beta regimen and were randomized in a 1:1:1 ratio to one of the following 3 treatment arms (see Table 1).

TABLE 1

| | | No. Total | No. |
|---|---|---|---|
| Treatment Arm¹ | Dose Level and Frequency | Dosing Visits | Patients |
| A (High Dose)² | Daclizumab SC: 2 mg/kg | 11 | 75 |
| | q2weeks × 11 doses | | |
| B (Low Dose)³ | Daclizumab SC: 1 mg/kg | 11 | 78 |
| | q4 weeks × 6 doses | | |
| C (Placebo)⁴ | Placebo SC: | 11 | 77 |
| | q2 weeks × 11 doses | | |

¹All patients continue on prior regimen of IFN-beta SC/IM for the duration of the study.
²Patients in Arm A (high dose) receive 2 SC injections (2 daclizumab 1 mg/kg) for 11 dosing visits. Maximum dose daclizumab per dosing visit = 200 mg.
³Patients in Arm B (low dose) receive 2 SC injections (1 daclizumab 1 mg/kg, 1 placebo) for 6 dosing visits, alternating with 2 SC injections (2 placebo) for 5 dosing visits. Maximum dose daclizumab per daclizumab dosing visit = 100 mg.
⁴Patients in Arm C (placebo) receive 2 SC injections (2 placebo) for 11 dosing visits.

The screening period was up to 3 weeks. The treatment period was designated as 24 weeks (6 months, through Day 168) in order to include 4 weeks subsequent to the last dose of blinded study drug (Dose No. 11, which occurs at Visit No. 14, Day 140). After the treatment period, patients were followed for a total of 48 weeks (12 months) and continued IFN beta therapy for at least 5 months of this period. Total maximum time on study for each patient was approximately 18 months.

Evaluations of a given patient by EDSS and Multiple Sclerosis Functional Composite, version 3 (MSFC-3) were performed by a clinician who was not involved in the patient's treatment and was designated an “evaluating clinician.” All other assessments of the patient were under the purview of the clinician in charge of the patient's treatment (treating clinician). The MSFC-3 includes quantitative tests of: (1) Leg function/ambulation—Timed 25-foot walk (T25FW); (2) Arm function—9-Hole Peg Test (9HPT), and (3) Cognition—Paced Auditory Serial Addition Test with 3-second interstimulus intervals (PASAT3) (Cutter et al., 1999, Brain, 122 (Pt 5):871-882).

Randomization was centralized and stratified by the dosing frequency of IFN beta (≧2 doses per week vs. <2 doses per week), EDSS score (0-2.0 and 2.5-5.0), and disease status (relapsing-remitting vs. secondary progressive).

Randomized patients received treatment with blinded study drug every 2 weeks, for a total of 20 weeks, for a total of 11 dosing visits per patient. Two SC injections were administered to each patient at each dosing visit, as presented in Table 2.

TABLE 2

| | Visit No. | | | | | | | | | | |
|---|---|---|---|---|---|---|---|---|---|---|---|
| | 2 | 5 | 6 | 7 | 8 | 9 | 10 | 11 | 12 | 13 | 14 |
| Week | 0 | 2 | 4 | 6 | 8 | 10 | 12 | 14 | 16 | 18 | 20 |
| Day | 0 | 14 | 28 | 42 | 56 | 70 | 84 | 98 | 112 | 126 | 140 |
| ±Days¹ | — | 4 | 4 | 4 | 4 | 4 | 4 | 4 | 4 | 4 | 4 |
| A² | DD | DD | DD | DD | DD | DD | DD | DD | DD | DD | DD |
| B³ | DP | PP | DP | PP | DP | PP | DP | PP | DP | PP | DP |
| C⁴ | PP | PP | PP | PP | PP | PP | PP | PP | PP | PP | PP |

¹±Days = Visit windows.
²A (high dose arm): Daclizumab 2 mg/kg q2 weeks × 11 doses. At each of the 11 dosing visits, patients receive 2 SC injections, each 1 mg/kg. Maximum dose per dosing visit = 200 mg.
³B (low dose arm): Daclizumab 1 mg/kg × q4 weeks × 6 doses. Patients receive 2 SC injections (1 daclizumab 1 mg/kg, 1 placebo) every 4 weeks for a total of 6 dosing visits, alternating with 2 SC placebo injections q4 weeks × 5, for a total of 5 dosing visits. Maximum dose per daclizumab dosing visit = 100 mg.
⁴C (placebo arm): 2 SC injections (each placebo) q2 weeks × 11 for a total of 11 dosing visits.
Abbreviations:
DD = 2 SC daclizumab injections,
DP = 2 SC injections, 1 daclizumab, 1 placebo,
PP = 2 SC placebo injections.

Patients were considered for inclusion in this study if they met all of the following criteria: (1) males and females, 18 to 55 years of age, inclusive; (2) diagnosis of MS by the McDonald criteria (see Table 3, below); (3) score <7.0 on the EDSS (described above); (4) on stable IFN-beta regimen, defined as at least 6 months on the same dose of the same drug product. Dose titration is allowed during the initial 2 months of IFN-beta treatment as long as the patient has remained on the adjusted dose for the remainder of the 6 month period. Patients who wish to participate must agree to remain on their same IFN beta regimen until Week 44 of the study; (5) the occurrence of either of the following within 12 months prior to screening: (a) at least one MS relapse while the patient was on a stable IFN-beta regimen or (b) a qualifying MRI, defined as an MRI that showed at least one confirmed gadolinium contrast-enhancing lesion (Gd-CEL) of the brain or spinal cord, performed while the patient was on a stable IFN-beta regimen; (6) for females, those who meet either of the following criteria: (a) non-childbearing potential, as documented by 1) surgical sterility from oophorectomy and/or hysterectomy or 2) a medical history (clinically or by follicle-stimulating-hormone testing) of being postmenopausal for at least the past 2 years. A history of tubal ligation, evidence of a spouse or sexual partner being sterile, or a history of sexual abstinence is insufficient evidence of non-childbearing potential; or (b) childbearing potential, provide a negative serum pregnancy test at screening and a negative urine pregnancy test within 24 hours of administration of first dose of study drug, and agree to utilize effective contraception or remain abstinent during the entire treatment and follow-up periods of the study; and (7) willing and able to comply with the protocol, provide informed consent in accordance with institutional and regulatory guidelines, and, for patients at US sites, authorization to use protected health information (HIPAA).

TABLE 3

| Clinical Presentation | Additional Data Needed |
|---|---|
| 2 or more attacks (relapses) | None; clinical evidence will suffice |
| 2 or more objective clinical | (additional evidence desirable but must be |
| lesions | consistent with MS) |
| 2 or more attacks | Dissemination in space, demonstrated by: |
| 1 objective clinical lesion | MRI or a positive CSF and 2 or more MRI |
| | lesions consistent with MS or further clinical |
| | attack involving different site |
| 1 attack | Dissemination in time, demonstrated by: |
| 2 or more objective clinical | MRI or second clinical attack |
| lesions | |
| 1 attack | Dissemination in space, by demonstrated by: |
| 1 objective clinical lesion | MRI |
| (monosymptomatic | or positive CSF and 2 or more MRI lesions |
| presentation) | consistent with MS and |
| | Dissemination in time, demonstrated by: |
| | MRI or second clinical attack |

Table 3 is adapted from McDonald, et al., 2001, Ann Neurol, 50: 121-127.

Patients were ineligible for enrollment in this study if they met any one of the following criteria: (1) pregnant or breast-feeding female; (2) non-ambulatory patient; (3) clinically significant abnormality on a screening electrocardiogram (ECG); (4) malignancy within the past 5 years except for adequately treated non-melanoma skin carcinoma or in situ carcinoma of the cervix; (5) a medical history of infection with the human immunodeficiency virus (HIV); (6) positive serology for infection with hepatitis B or C virus (HBV or HCV); (7) varicella or herpes zoster virus infection or any severe virus infection within 6 weeks before screening; (8) exposure to varicella zoster virus within 21 days before screening; (9) abnormal hematology, defined as any of the following: Hemoglobin ≦8.5 g/dL; Lymphocytes ≦1.0×109/L; Platelets ≦100×109/L; Neutrophils ≦1.5×109/L; (10) significant organ dysfunction, including but not limited to cardiac, renal, liver, non-MS related CNS, pulmonary, vascular, gastrointestinal, endocrine, or metabolic dysfunction, or other disease or condition, which in the opinion of the principal investigator would make the patient an unsuitable candidate for the study. Guidelines for levels of unacceptable dysfunction include: creatinine ≧1.6 mg/dL; AST and ALT ≧2.5 upper limit of normal [ULN]; alkaline phosphatase ≧2.5 ULN; history of myocardial infarction, congestive heart failure, or arrhythmias within 6 months prior to randomization; (11) use of any of the following: (a) any of the following types of live virus vaccine from 4 weeks before randomization: measles/mumps/rubella vaccine, varicella zoster virus vaccine, oral polio vaccine, and nasal influenza vaccine. Use of these vaccines, however, by household contacts does not affect the eligibility of patients to enroll or continue in the study; (b) systemic corticosteroids, adrenocorticotropic hormone, or plasma exchange within 4 weeks before the baseline MRI scan (no more than 72 hours before Day 0); (c) azathioprine, mycophenolate mofetil, methotrexate, glatiramer acetate, or intravenous immune globulin within 6 months before randomization; (d) an immunomodulatory agent within 6 months before randomization, except for interferon-beta products required per protocol; (e) an investigational agent within 6 months before randomization unless this agent is non-immunomodulatory and the medical monitor or steering committee rules that its use is acceptable on the theoretical basis of a lapse of at least 5 serum half-lives since administration of the last possible dose; (f) a monoclonal antibody (eg, Rituxan®/Rituximab) within 6 months before randomization; (g) daclizumab at any time prior to randomization; (h) cladribine, mitoxantrone, cyclophosphamide, CamPath® (alemtuzumab), natalizumab (TYSABRI®/Antegren) or other drugs targeting alpha 4 integrin, total lymphoid irradiation, or bone marrow transplant at any time; (i) illegal drugs of abuse, for example, marijuana, cocaine, and lysergic acid (LSD); (12) patients for whom MRI is contraindicated, ie, have pacemakers or other contraindicated implanted metal devices, are allergic to gadolinium, or have claustrophobia that cannot be medically managed; (13) primary progressive MS; (14) clinically unstable for 30 days before randomization (Patients who experience a relapse, with or without steroid treatment, during the screening period may be re-screened after 30 days); (15) elective surgery performed from 2 weeks prior to randomization or scheduled through Week 44; and (16) infection (viral, fungal, bacterial) requiring hospitalization or IV antibiotics within 8 weeks before randomization.

Preliminary eligibility for the CHOICE study was established by history, chart inspection, and routine evaluations. During the treatment and follow up period, a number of procedures and evaluations were performed on the subjects at specified days including, but not limited to, MRI, EDSS, MSFC-3, physical exams, symptom directed physical exams, hematology/serum chemistry (e.g., for determination of pharmacokinetic assessment and anti-DAC antibodies), and blood draws for pharmacodynamic assessments and IFN-beta NAbs.

Daclizumab drug substance manufactured by PDL BioPharma, Inc. (Redwood City, Calif.) for subcutaneous delivery, was supplied in single-use vials containing 100 mg of daclizumab in 1.0 mL of 40 mM sodium succinate, 100 mM sodium chloride, 0.03% polysorbate 80, pH 6.0. Placebo was supplied in single-use vials as an isotonic solution in matching vials containing 40 mM sodium succinate, 6% sucrose, 0.03% polysorbate 80, pH 6.0.

### Example 2

### Treatment of MS IFN-Beta NAb Positive Subjects with Daclizumab

A subset of the patients in the CHOICE study developed neutralizing antibodies to IFN-beta. The efficacy of daclizumab was evaluated in this subset of subjects who were positive for IFN-beta neutralizing antibodies (NAb) during the DAC dosing period in the CHOICE study.

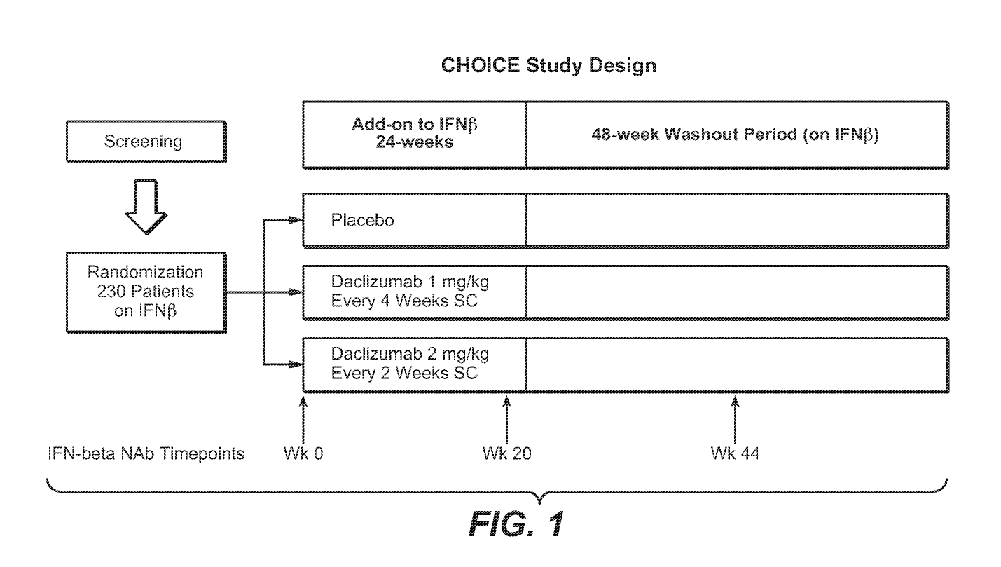

Timepoints for the collection of blood to analyze for the presence of IFN-betaNAbs are shown in FIG. 1. IFN-beta NAb were detected using a standard cell-based viral inhibition assay. The methodology followed the guidelines of the NIH Committee on Human Antibodies to Interferon and the World Health Organization for the standardization of interferon neutralization bioassays. The assay was performed by Athena Diagnostics, Worcester, Mass. Results for serum samples are given as titer values (reciprocal dilution of serum which reduces interferon activity by a standard amount). A titer of >20 was considered positive. A subject was defined as positive for IFN-beta NAb if the titer of both the Week 0 (pre-dose) and Week 20 samples was >25. These time points bracketed the DAC dosing period.

Statistical methods: A negative binomial regression model was used to compare the total number of new or enlarged Gd-CELs (Weeks 8 to 24) between each active group and placebo. The model for the primary efficacy analysis was adjusted for both the baseline number of Gd-CELs and baseline disease status (relapsing remitting MS or secondary progressive MS). The model for analysis in the IFN-beta NAb positive subgroup was adjusted for baseline number Gd-CELs only. Due to the small sample size in the subgroup, results were confirmed using a Kruskal-Wallis test.

The overall incidence of IFN-beta NAB positive subjects in the CHOICE study is shown in Table 4. Only subjects for whom data was collected at week 0 and 20 were included in the efficacy analysis of the IFN-beta NAb subgrouped subjects.

TABLE 4

| | | IFN-beta NAb | IFN-beta NAb |
|---|---|---|---|
| Treatment Group | N | Positive Subjects | Negative Subjects |
| 2 mg/kg q2wk SC | 65 | 7 (11%) | 58 |
| 2 mg/kg q4wk SC | 63 | 7 (11%) | 56 |
| Placebo | 68 | 9 (13%) | 59 |

Titer values for the IFN-beta NAb positive subjects is shown in Table 5.

TABLE 5

| Dosage | Subject | | | |
|---|---|---|---|---|
| Group | No. | Week 0 | Week 20 | Week 40 |
| 2 mg/kg | 1 | >640 | 218 | >640 |
| | 2 | 69 | 97 | >640 |
| | 3 | 85 | 46 | 170 |
| | 4 | 45 | 132 | 87 |
| | 5 | 36 | 39 | <20 |
| | 6 | 80 | 71 | <20 |
| | 7 | 100 | 54 | <20 |
| 1 mg/kg | 8 | 145 | 157 | 143 |
| | 9 | >640 | >640 | >640 |
| | 10 | 140 | 125 | 45 |
| | 11 | >640 | >640 | >640 |
| | 12 | 140 | 193 | 257 |
| | 13 | 180 | 157 | 416 |
| | 14 | >640 | >640 | >640 |
| Placebo | 15 | >640 | >640 | >640 |
| | 16 | >640 | >640 | >640 |
| | 17 | 52 | 68 | >640 |
| | 18 | 84 | 291 | 74 |
| | 19 | >640 | >640 | >640 |
| | 20 | 139 | 136 | >640 |
| | 21 | >640 | >640 | >640 |
| | 22 | 58 | 27 | 30 |
| | 23 | 65 | >640 | No sample |

A subject was defined as positive for IFN-beta NAb if the titer of both the Week 0 (pre-dose) and Week 20 samples was >25. Week 44 timepoints were analyzed but not used to define IFN-beta NAb positivity. Data are provided to show that for most subjects, high titer values at Week 20 remained high at Week 44. A titer value of 25 was chosen as the cut-off to increase the chances that the response was not only positive (>20), but would also result in a decrease of exposure to IFN-beta.

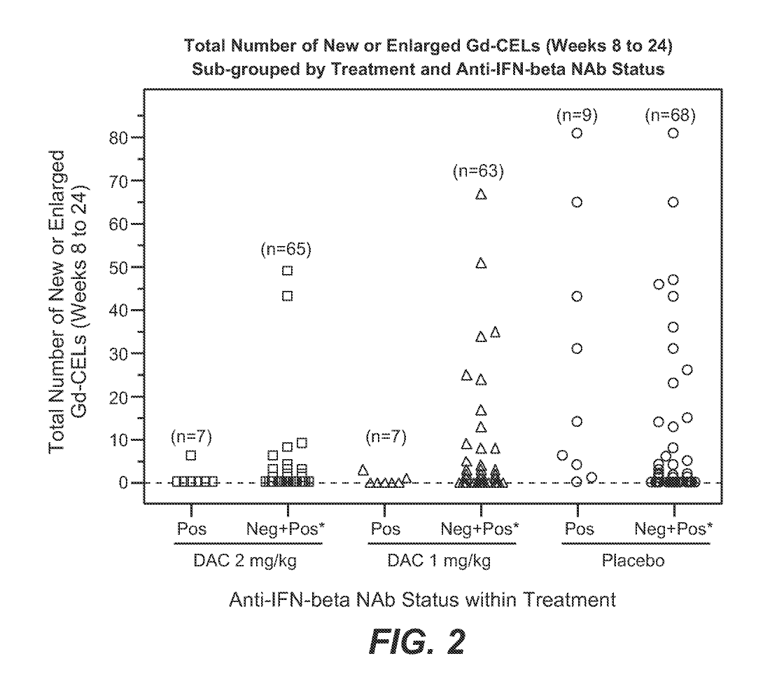

FIG. 2 is a scatter plot showing the relationship between number of new or enlarged Gd-CELs (Weeks 8 to 24) versus treatment and IFN-beta NAb status. Placebo subjects show higher numbers of new or enlarged Gd-CELs compared with both 2 mg/kg and 1 mg/kg DAC treated subjects. For both DAC treated groups, the IFN-beta NAb positive subjects are associated with low numbers of new or enlarged Gd-CELs, from Weeks 8 to 24.

The primary efficacy analysis for the intent to treat (ITT) population and IFN-beta NAb positive subjects is shown in Table 6.

TABLE 6

| | | Total Number of Gd-CELs | | |
|---|---|---|---|---|
| | | (new or enlarged) | | |
| Analysis | | | Adj. Mean^{a} | P value vs |
| Population | Treatment | N | (SE) | Placebo^{b} |
| Intent-to- | Placebo | 76 | 4.75 (1.99) | |
| Treat^{c} | DAC 1 mg/kg | 78 | 3.58 (1.57) | 0.514 |
| | DAC 2 mg/kg | 75 | 1.32 (0.55) | 0.004 |
| IFN-beta NAb | Placebo | 9 | 20.76 (11.40) | |
| positive | DAC 1 mg/kg | 7 | 0.32 (0.32) | 0.0002 |
| | DAC 2 mg/kg | 7 | 1.21 (0.99) | 0.0043 |

^{a}Adjusted means are obtained using a negative binomial model; SE = standard error.
^{b}p values obtained from the differences of the least square means between compared treatment groups.
^{c}ITT includes all subjects who were randomized and received any study drug.

In Table 6, N differs from intent-to-treat (ITT) population since only subjects with available Week 0 and Week 20 IFN-beta NAb data were considered for IFN-beta NAb population. For the ITT (intent-to-treat) population, the primary endpoint (total new or enlarged Gd-CELs from Weeks 8 to 24) was significant (p=0.004) for the 2 mg/kg group, but not for the 1 mg/kg group (p=0.514) when compared with placebo. For IFN-beta NAb positive subjects, significantly lower numbers of new or enlarged Gd-CELs were obtained for both the 2 mg/kg group (p=0.0043) and 1 mg/kg group (p=0.0002) when compared with placebo.

The mean number of total new or enlarged Gd-CELs in IFN-beta NAb positive and negative subjects is shown in Table 7.

TABLE 7

| | N | DAC 1 mg/kg | N | DAC 2 mg/kg |
|---|---|---|---|---|
| IFN-beta NAb | 7 | 0.32 (0.32)^{a} | 7 | 1.21 (0.99) |
| positive | | | | |
| IFN-beta NAb | 56 | 4.97 (1.72) | 58 | 1.39 (0.49) |
| negative | | | | |

^{a}Data presented as adjusted mean with standard error in paranthesis.

The mean number of total new or enlarged Gd-CELs was lower in subjects positive for IFN-beta NAb compared to subjects who were negative for IFN-beta NAb. The result was a post-study observation, and therefore does not warrant any formal statistical comparison of the two groups.

One potential concern with the statistical analysis using the negative binomial model was the small sample size of IFN-beta NAb positive subjects. Therefore, the Kruskal-Wallis test was used as an alternative statistical analysis method, with the following results for IFN-beta NAb positive subjects: DAC 2 mg/kg vs. placebo, p=0.0057; and DAC 1 mg/kg vs. placebo, p=0.0055. These results are consistent with those obtained based on the negative binomial model.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

### SPECIFIC EMBODIMENTS, CITATION OF REFERENCES

The methods described herein can be illustrated by the following embodiments enumerated in the numbered paragraphs that follow:

1. A method of treating a human patient with multiple sclerosis (“MS”), comprising: administering a therapeutically effective amount of an anti-IL-2R antibody to said patient, thereby ameliorating a symptom of multiple sclerosis and treating the patient.

2. The method according to paragraph 1, wherein the patient has received interferon beta (“IFN-beta”) therapy prior to said treatment.

3. The method according to paragraph 2, wherein the patient is refractory to IFN-beta therapy.

4. The method according to paragraph 2 or paragraph 3, wherein the patient does not receive IFN-beta therapy concurrently with said administration.

5. The method according to paragraph 2 or paragraph 3, wherein the patient receives IFN-beta therapy concurrently with said administration.

6. The method according to any one of paragraphs 2 to 5 wherein the patient is positive for neutralizing antibodies to IFN-beta.

7. The method according to paragraph 6, wherein the patients has a IFN-beta neutralizing antibody titer greater than 20 in two or more consecutive samples.

8. The method according to paragraph 6, wherein the patient has a IFN-beta neutralizing antibody titer greater than 25 in two or more consecutive samples.

9. The method according to paragraph 1, wherein the patient is negative for neutralizing antibodies to IFN-beta.

10. The method according to any one of paragraphs 1 to 8, wherein ameliorating a symptom of multiple sclerosis comprises reducing the number of relapses in a given period.

11. The method according to any one of paragraphs 1 to 10, wherein ameliorating a symptom of multiple sclerosis comprises reducing the rate of increase of the patient's Expanded Disability Status Score.

12. The method according to paragraph 1 to 11, wherein ameliorating a symptom of multiple sclerosis comprises reducing the number of T1 gadolinium contrast-enhanced MRI lesions.

13. The method according to paragraph 1 to 12, wherein ameliorating a symptom of multiple sclerosis comprises reducing the number of T2 gadolinium contrast-enhanced MRI lesions.

14. The method according to any one of paragraphs 1 to 13, wherein the anti-IL-2R antibody binds to IL-2R subunit CD25.

15. The method according to paragraph 14, wherein the antibody competes with daclizumab for binding to CD25.

16. The method according to paragraph 15, wherein the anti-IL2R antibody is basilixmab.

17. The method according to paragraph 15, wherein the anti-IL2R antibody is daclizumab.

18. The method according to paragraph 17, wherein the patient is positive for neutralizing antibodies to IFN-beta and wherein daclizumab is administered at a dose of about 0.5 to about 5 milligrams per kilogram of said patient's body weight.

19. The method according to paragraph 18, wherein daclizumab is administered at a dose of about 0.5 to about 1.5 milligrams per kilogram.

20. The method according to paragraph 19, wherein daclizumab is administered at a dose of about 1 to about 1.5 milligrams per kilogram.

21. The method according to paragraph 18, wherein daclizumab is administered at a dose of about 1 to about 2 milligrams per kilogram.

22. The method according to paragraph 18, wherein daclizumab is administered at a dose of about 2 to about 4 milligrams per kilogram.

23. The method according to any one of paragraphs 18 to 22, wherein the patient has a IFN-beta neutralizing antibody titer greater than 20 in two or more consecutive samples.

24. The method according to any one of paragraphs 18 to 22, wherein the patient has a IFN-beta neutralizing antibody titer greater than 25 in two or more consecutive samples.

25. The method according to paragraph 17, wherein the patient is negative for neutralizing antibodies to IFN-beta and wherein daclizumab is administered at a dose of about 1 to about 7.5 milligrams per kilogram.

26. The method according to paragraph 25, wherein daclizumab is administered at a dose of about 1 to about 5 milligrams per kilogram.

27. The method according to paragraph 26, wherein daclizumab is administered at a dose of about 2 to about 4 milligrams per kilogram.

28. The method according to any one of paragraphs 25 to 27, wherein daclizumab is administered at a dose of greater than about 1.5 milligrams per kilogram.

29. The method according to any one of paragraphs 25 to 27, wherein daclizumab is administered at a dose of greater than about 2 milligrams per kilogram.

30. The method according to any one of paragraphs 1 to 29, wherein said anti-IL2R antibody is administered intravenously.

31. The method according to any one of paragraphs 1 to 29, wherein said anti-IL2R antibody is administered subcutaneously.

32. The method according to any one of paragraphs 1 to 31, wherein said anti-IL2R antibody is administered at least biweekly.

33. The method according to any one of paragraphs 1 to 31, wherein said anti-IL2R antibody is administered at least monthly.

34. The method according to any one of paragraphs 1 to 31, wherein said anti-IL2R antibody is administered every 4 weeks.

35. The method according to any one of paragraphs 1 to 31, wherein said anti-IL2R antibody is administered every 5 weeks.

36. The method according to any one of paragraphs 1 to 31, wherein said anti-IL2R antibody is administered every 6 weeks.

37. The method according to any one of paragraphs 1 to 31, wherein said anti-IL2R antibody is administered every 7 weeks.

38. The method according to any one of paragraphs 1 to 31, wherein said anti-IL2R antibody is administered every 8 weeks.

39. The method according to any one of paragraphs 1 to 38, wherein the patient has relapsing-remitting multiple sclerosis.

40. The method according to any one of paragraphs 1 to 38, wherein the patient has secondary progressive multiple sclerosis.

41. The method according to any one of paragraphs 1 to 38, wherein the patient has progressive relapsing multiple sclerosis.

42. The method according to any one of paragraphs 1 to 38, wherein the patient has primary progressive multiple sclerosis.

43. A method of treating multiple sclerosis in a human patient, comprising:
(a) testing the patient for the presence of IFN-beta neutralizing antibodies; and
(b) administering to said patient a dose of daclizumab, wherein the dose is greater than about 1.25 milligrams per kilogram of said patient's body weight if the patient is negative for IFN-beta neutralizing antibodies and less then about 1.25 milligrams per kilogram of said patient's body weight if said patient is positive for IFN-beta neutralizing antibodies.

44. The method of paragraph 43, wherein the dose is about 2 milligrams per kilogram of said patient's body weight if the patient is negative for IFN-beta neutralizing antibodies.

45. The method of paragraph 43 or 44, wherein the dose is about 1 milligram per kilogram of said patient's body weight if the patient is positive for IFN-beta neutralizing antibodies.

46. A method of stratifying a MS patient population into at least levels of intervention, comprising:
(a) testing the patient population for the presence of IFN-beta neutralizing antibodies;
(b) stratifying those patients into at least a first population and a second population, wherein the first population is negative for IFN-beta neutralizing antibodies and the second population is positive for IFN-beta neutralizing antibodies; and
(c) administering an anti-IL-2R antibody to said first population and to second population, wherein the dose administered to said first population is higher than the dose administered to said second population and/or wherein said anti-IL-2R antibody is administered at a higher frequency to the first population than to the second population and/or wherein daclizumab is administered over a longer period to the first population than to the second population.

47. The method according to paragraph 46, wherein said anti-IL-2R antibody is daclizumab.

48. The method according to paragraph 46, wherein the dose administered to said first population is higher than the dose administered to said second population.

49. The method of paragraph 48, wherein anti-IL-2R antibody is daclizumab and the dose administered to said first population is 1.5 to 2.5 milligrams per kilogram of each patient's body weight and where in the dose administered to said second population is 0.8 to 1.5 milligrams per kilogram of each patient's body weight.

50. The method of any one of paragraphs 46 through 49, wherein anti-IL-2R antibody is administered at a higher frequency to the first population than to the second population.

51. The method of paragraph 50, wherein anti-IL-2R antibody is administered to said first population at an average of every two weeks and to said second population at an average of every four weeks.

52. The method of any one of paragraphs 46 through 51, wherein said anti-IL-2R antibody is administered to said a first population over a treatment period that is greater than the treatment period over which daclizumab is administered to said second population.

What is claimed is:

1. A method of treating MS patients that have neutralizing antibodies to interferon beta comprising, administering a therapeutically effective amount of anti-IL-2R antibody to the patients, thereby ameliorating a symptom of multiple sclerosis and treating the subject.

2. The method according to claim 1, wherein the MS patients have a IFN-beta neutralizing antibody titer greater than 20 in two or more consecutive samples.

3. The method according to claim 1, wherein the MS patients have a IFN-beta neutralizing antibody titer greater than 25 in two or more consecutive samples.

4. The method according to claim 1, wherein the anti-IL-2R antibody is daclizumab.

5. The method according to claim 1, wherein ameliorating a symptom of multiple sclerosis comprises reducing the number of relapses in a given period.

6. The method according to claim 1, wherein ameliorating a symptom of multiple sclerosis comprises reducing the rate of increase of the subject's Expanded Disability Status Score.

7. The method according to claim 1, wherein ameliorating a symptom of multiple sclerosis comprises reducing the number of T1 gadolinium contrast-enhanced MRI lesions.

8. The method according to claim 1, wherein ameliorating a symptom of multiple sclerosis comprises reducing the number of T2 gadolinium contrast-enhanced MRI lesions.

9. The method of claim 1, wherein daclizumab is administered at a dose of about 0.5 to about 5 milligrams per kilogram.

10. The method of claim 5, wherein daclizumab is administered at a dose of about 0.5 to about 1.5 milligrams per kilogram.

11. The method of claim 5, wherein daclizumab is administered at a dose of about 1 to about 1.5 milligrams per kilogram.

12. The method of claim 5, wherein daclizumab is administered at a dose of about 1 to about 2 milligrams per kilogram.

13. The method of claim 5, wherein daclizumab is administered at a dose of about 2 to about 4 milligrams per kilogram.

14. The method of claim 5, wherein daclizumab is administered intravenously.

15. The method of claim 5, wherein daclizumab is administered subcutaneously.

16. The method of claim 1, wherein daclizumab is administered at least biweekly.

17. The method of claim 1, wherein daclizumab is administered at least monthly.

18. The method of claim 1, wherein the subject has relapsing-remitting multiple sclerosis.

19. The method of claim 1, wherein the subject has secondary progressive multiple sclerosis.

20. The method of claim 1, wherein the subject has progressive relapsing multiple sclerosis.

21. The method of claim 1, wherein the subject has primary progressive multiple sclerosis.

22. A method of treating an MS patient, the method comprising
a. determining whether a biological sample from an MS patient contains neutralizing interferon beta antibody; and
b. if the biological sample contains neutralizing interferon beta antibody, administering to the patient a therapeutically effective amount of anti-IL-2R antibody, thereby ameliorating at least one symptom of multiple sclerosis.

23. The method according to claim 22, wherein the MS patient has an IFN-beta neutralizing antibody titer greater than 20 in two or more consecutive samples or the MS patient has an IFN-beta neutralizing antibody titer greater than 25 in two or more consecutive samples.

24. The method according to claim 22, wherein the anti-IL-2R antibody is daclizumab.

25. The method according to claim 22, wherein ameliorating a symptom of multiple sclerosis comprises reducing the number of relapses in a given period, reducing the rate of increase of the subject's Expanded Disability Status Score, reducing the number of T1 gadolinium contrast-enhanced MRI lesions, or reducing the number of T2 gadolinium contrast-enhanced MRI lesions.

26. The method of claim 24, wherein daclizumab is administered at a dose of about 0.5 to about 5 milligrams per kilogram, daclizumab is administered at a dose of about 0.5 to about 1.5 milligrams per kilogram, daclizumab is administered at a dose of about 1 to about 1.5 milligrams per kilogram, daclizumab is administered at a dose of about 1 to about 2 milligrams per kilogram, or daclizumab is administered at a dose of about 2 to about 4 milligrams per kilogram.

27. The method of claim 24, wherein daclizumab is administered intravenously.

28. The method of claim 24, wherein daclizumab is administered subcutaneously.

29. The method of claim 22, wherein daclizumab is administered at least biweekly or at least monthly.

30. The method of claim 22, wherein the subject has relapsing-remitting multiple sclerosis, progressive relapsing multiple sclerosis, or primary progressive multiple sclerosis.
